(19) United States
(12) Patent Application Publication
(10) Pub. No.: US 2024/0293236 A1
(43) Pub. Date: Sep. 5, 2024

(54) SPINAL SURGICAL APPROACH METHOD

(71) Applicant: NovApproach Spine, LLC, Alachua, FL (US)

(72) Inventors: Michael MacMillan, Gainesville, FL (US); Raymond Cloutier, Alachua, FL (US)

(21) Appl. No.: 17/808,538

(22) Filed: Jun. 23, 2022

Related U.S. Application Data
(60) Provisional application No. 63/214,199, filed on Jun. 23, 2021.

(51) Int. Cl. A61F 2/46; A61F 2/44

(52) U.S. Cl. CPC A61F 2/4611; A61F 2/442; A61F 2/4455

(57) ABSTRACT

The present invention provides a spinal surgical approach which provides approaching the spine of a patient for spinal interbody fusion or total disc replacement through the oblique (a.k.a. anterior to psoas) corridor in an improved manner. Aspects of the invention further provide a means to facilitate the restoration of sagittal plane lordotic alignment. Other aspects of the invention provide a means to facilitate exposure of the spine and shorten operative times with fewer steps. In certain embodiments, aspects of the invention provide for improved instrumentation and devices to facilitate the surgical approach method so that it can be accomplished in a simpler and more effective manner.

### CROSS-REFERENCES TO RELATED APPLICATIONS

This invention is a continuation in part of, and claims priority to U.S. Provisional Application for Patent No. 63/214,199, which was filed on Jun. 23, 2021, the entirety of the foregoing references are incorporated herein by this reference.

### FIELD OF INVENTION

The present invention generally relates to spinal surgical procedures in a human. More particularly, the disclosure generally relates to surgical methods for use with spinal implantation approaches.

### BACKGROUND

The treatment of disorders of the spine has advanced over the years to be adapted to address many forms of spinal disorders. Multiple surgical approaches exist to treat spinal disorders, including Anterior Lumbar Interbody Fusion (a.k.a. ALIF), Oblique Lumbar Interbody Fusion/Anterior to Psoas (a.k.a. OLIF/ATP), Supine ATP and Direct Lateral Interbody Fusion/Extreme Lateral Interbody Fusion (a.k.a. DLIF, XLIF), to name a few.

U.S. patent application Ser. No. 17/444,084 provides one method of approaching the spine of a patient for spinal interbody fusion or total disc replacement through the oblique corridor, which enables the patient to be positioned on the operating table in the supine position. However, that method does not provide a means to facilitate the restoration of sagittal plane lordotic alignment nor does it provide various means to facilitate exposure of the spine and shorten operative times with fewer steps. Thus, there remains an unmet need for approaching the spine of a patient for spinal interbody fusion or total disc replacement through the oblique corridor in an improved manner.

In addition, there remains an unmet need for improved instrumentation and devices to facilitate the surgical approach method so that it can be accomplished in a simpler and more effective manner.

### SUMMARY OF INVENTION

The present invention provides a surgical implant approach which provides approaching the spine of a patient for spinal interbody fusion or total disc replacement through the oblique corridor in an improved manner.

Aspects of the invention further provide a means to facilitate the restoration of sagittal plane lordotic alignment.

Other aspects of the invention provide a means to facilitate exposure of the spine and shorten operative times with fewer steps.

While yet, in certain embodiments, aspects of the invention provide for improved instrumentation and devices to facilitate the surgical approach method so that it can be accomplished in a simpler and more effective manner.

### BRIEF DESCRIPTION OF THE DRAWINGS

Examples illustrative of embodiments of the disclosure are described below with reference to figures attached hereto. In the figures, identical structures, elements or parts that appear in more than one figure are generally labeled with the same numeral in all the figures in which they appear. Dimensions of components and features shown in the figures are generally chosen for convenience and clarity of presentation and are not necessarily shown to scale. Many of the figures presented are in the form of schematic illustrations and, as such, certain elements may be drawn greatly simplified or not-to-scale, for illustrative clarity. The figures are not intended to be production drawings. The figures (Figs.) are listed below.

FIG. 1 illustrates the orientation of the ALIF, AL-ALIF, and Supine ATP surgical approach methods relative to a Subject's vertebrae.

FIG. 2 shows a method of surgically approaching the spine of a patient for spinal intervertebral implantation with a lumbar support.

FIG. 3 shows a method of surgically approaching the spine of a patient for spinal intervertebral implantation with a buttock bolster.

FIG. 4 shows a method of surgically approaching the spine of a patient for spinal intervertebral implantation with a torso tilt.

FIG. 5 shows a method of surgically approaching the spine of a patient for spinal intervertebral implantation with a lumbar support, buttock bolster, and torso tilt.

FIG. 6 shows a subject in position with a flattened spine.

FIG. 7 shows a subject in position with a lumbar support (e.g. roll) and a lordotic spine.

FIG. 8 shows a peritoneal sac of a subject in a level position.

FIG. 9 shows a mobilized sac of a subject in a tilted position.

FIG. 10 shows another exemplary embodiment that maintains position without the need for a retractor frame.

FIG. 11 shows a method of surgically approaching the spine of a patient for spinal intervertebral implantation with an angled patient positioner.

FIG. 12 shows a method of surgically approaching the spine of a patient for spinal intervertebral implantation with the patient in the tilted position during the final steps of the procedure.

FIG. 13 shows a method of surgically approaching the spine of a patient for spinal intervertebral implantation without the need for any OR table rotation.

FIG. 14 shows a subject in a first position on an angled patient positioner on an OR table that is oriented parallel to the floor.

FIG. 15 shows a subject in a second position on an angled patient positioner on an OR table that is oriented at an angle to the floor.

FIG. 16a illustrates a patient experiencing difficulty standing upright due to being fused with a flattened spine.

FIG. 16b illustrates a patient restored to normal posture due a realignment operation to restore lumbar lordosis.

FIG. 17 illustrates that a smaller cross section of the psoas muscle can be obtained based on a subject's position. A smaller cross section of the psoas reduces the risk of iatrogenic injury to this muscle during the development of the spinal approach corridor.

FIG. 18 illustrates body planes showing a patient and exemplary axes.

FIG. 19 shows exemplary supine, right lateral recumbent, and prone positions of an exemplary patient.

FIG. 20 shows a subject in position with a buttock bolster and the effect this position has on reducing the psoas muscle width.

FIG. 21 shows a subject in position without a buttock bolster showing a loose psoas muscle.

It should be clear that the description of the embodiments and attached Figures set forth in this specification serves only for a better understanding of the disclosure, without limiting its scope. It should also be clear that a person skilled in the art, after reading the present specification could make adjustments or amendments to the attached Figures and above described embodiments that would still be covered by the present disclosure.

### DETAILED DESCRIPTION

This disclosure describes exemplary medical methods, apparatus, systems, etc. for use in spinal surgical procedures. This disclosure further provides for use of medical devices across multiple spinal surgical approaches.

It is to be understood that the present disclosure is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

Unless defined otherwise, all technical and scientific terms used herein have the same meaning as commonly understood by one of ordinary skill in the art.

The phrase “ATP”, as used herein, generally refers to a surgical pathway that is anterior to the psoas muscle in a Subject including, but not limited to, the pathway anterior to the psoas muscle in the abdomen.

The phrase ′AL″, as used herein, generally refers to the surgical pathway that is lateral to a direct anterior-midline surgical pathway in a Subject including, but not limited to the retroperitoneal pathway to the spine in the abdomen.

The phrase ‘Cage’, as used herein, generally refers to a spacer (a.k.a. “interbody”) implant or bone graft that is positioned between adjacent vertebrae during spinal surgery.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

The present disclosure provides for a plurality of surgical methods.

### Surgical Methods

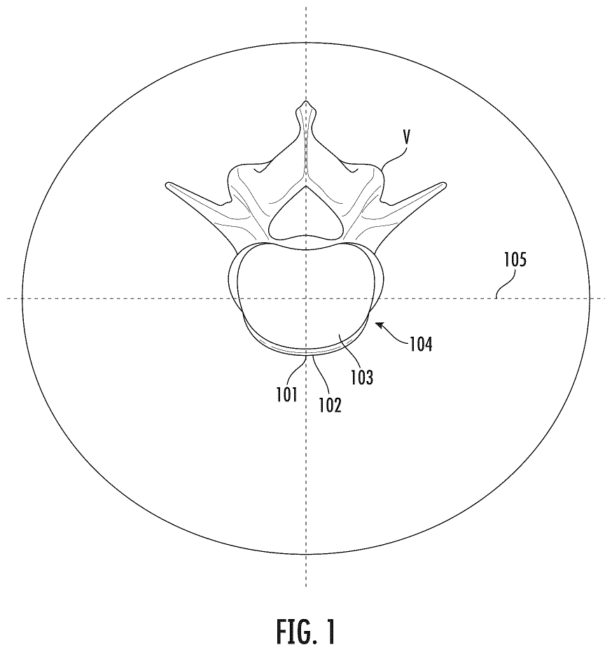

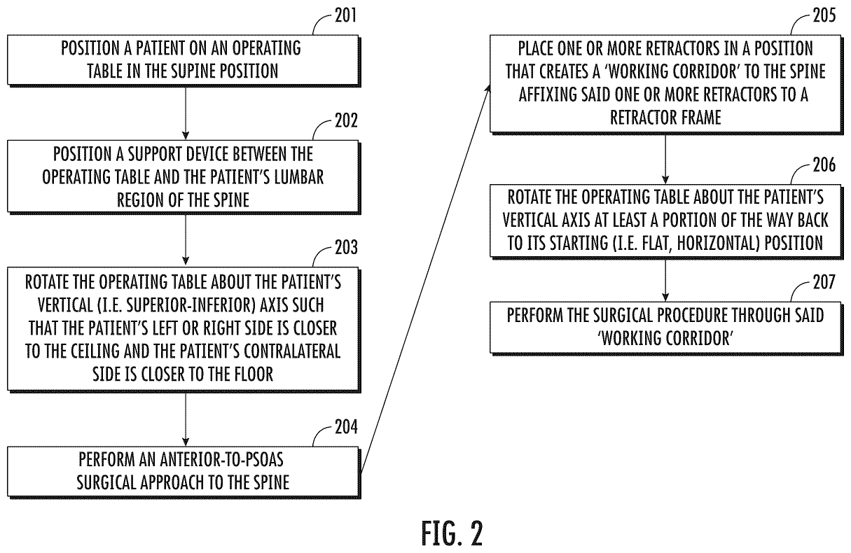

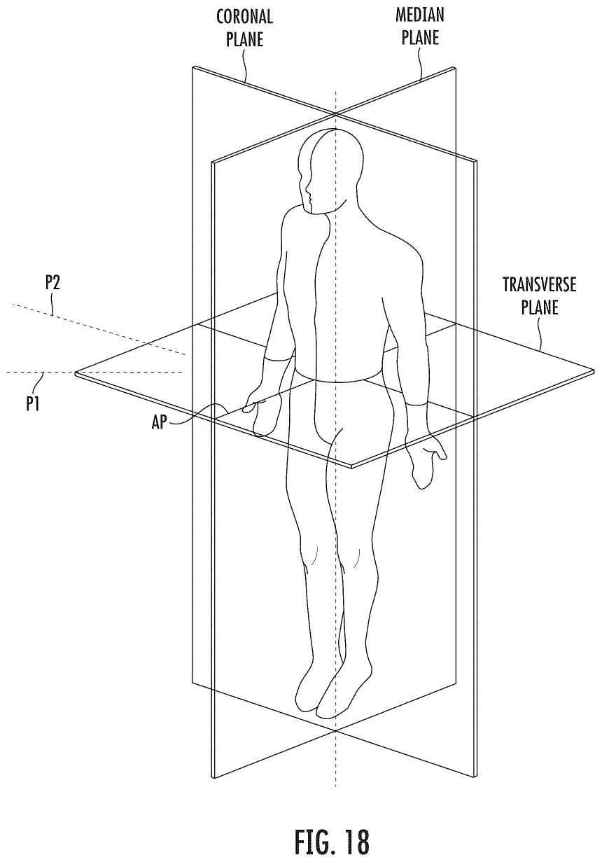

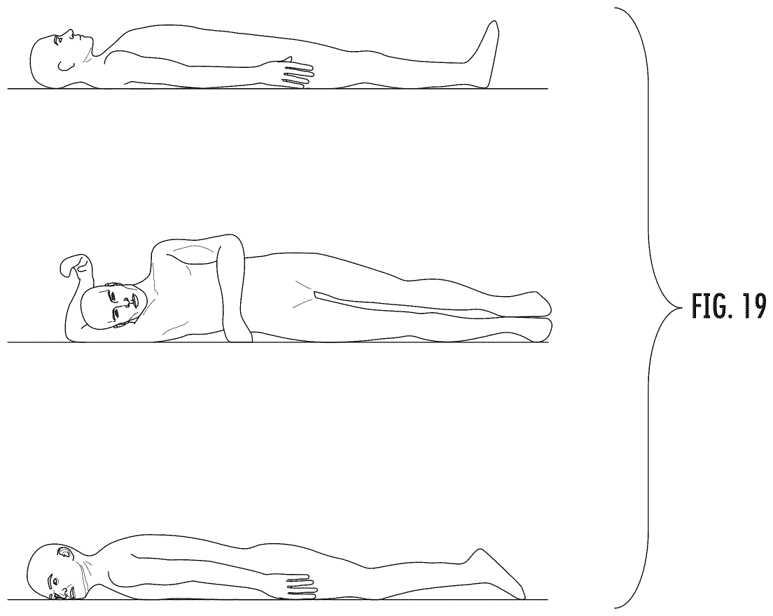

Embodiments of the disclosure allow for a plurality of surgical methods including, without limit, anterior, anterior-lateral, oblique, anterior to psoas, lateral, or combinations thereof. For reference, FIG. 1 shows exemplary orientations of ALIF at 101, AL-ALIF at 102, and ATP, Supine-ATP, and Supine Oblique at 103 and 104 surgical approaches relative to a Subject's vertebrae V. An exemplary right/left axis in the transverse plane is also shown at 105. For further reference, FIG. 18 illustrates body planes showing a patient with exemplary axes. FIG. 19 shows exemplary supine, right lateral recumbent, and prone positions of an exemplary patient. In addition, FIGS. 2-15 illustrate various uses with surgical methods and examples of use.

Supine ATP with Lumbar Support Method

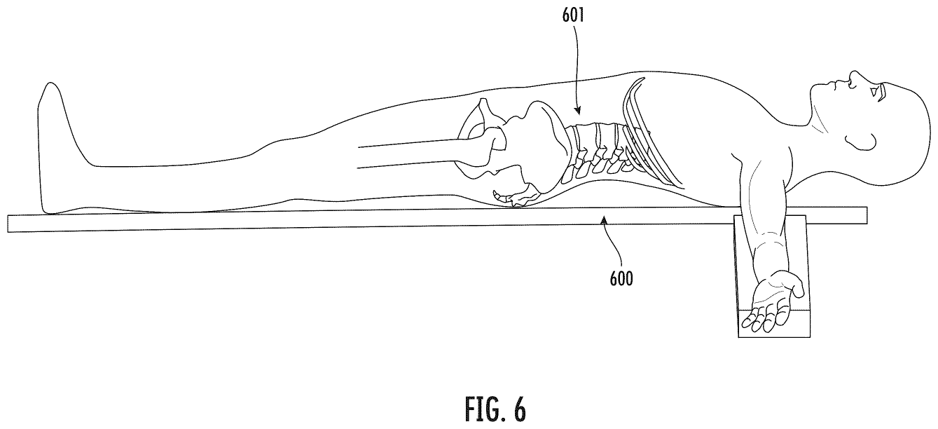

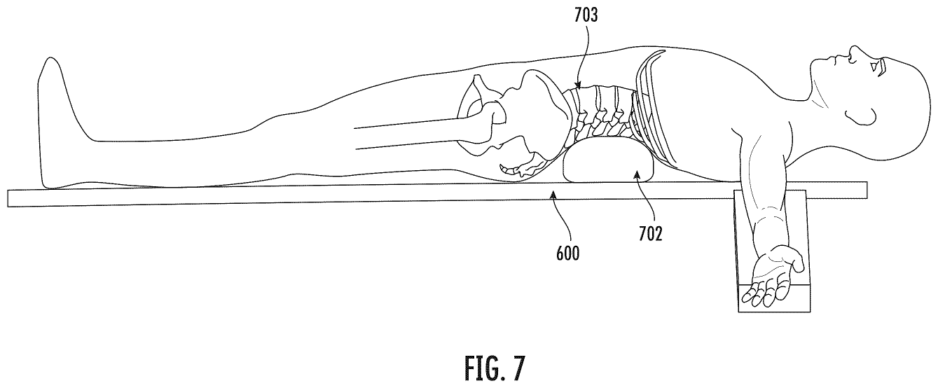

Embodiments of the present disclosure allow for inventive surgical methods to be used in association with one or more spinal interbody fusion or disc replacement device. It should be appreciated that such methods may be used with any spinal interbody fusion or disc replacement device, and nothing herein is intended to limit the surgical methods strictly to the use of the spinal interbody fusion devices disclosed herein. FIG. 2 illustrates at least one method of surgically approaching the spine of a patient for spinal intervertebral implantation with a lumbar support. FIG. 6 illustrates a patient positioned on an 600 operating table without any lumbar support illustrating a 601 flat spine. FIG. 7 illustrates a patient positioned on an 600 operating table with a 702 lumbar support device (e.g. roll) and a 703 lordotic spine. As illustrated in FIG. 2, in at least one embodiment, the surgical method includes 201 positioning a patient on an operating table in the supine position, 202 positioning a support device between the operating table and the patient's lumbar region of the spine, 203 rotating the operating table about the patients' vertical (i.e. superior-inferior) axis such that the patient's left or right side is closer to the ceiling and the patient's contralateral side is closer to the floor, 204 performing an anterior-to-psoas surgical approach to the spine, 205 placing one or more retractors in a position that creates a ‘working corridor’ to the spine affixing said one or more retractors to a retractor frame, 206 rotating the operating table about the patient's vertical axis at least a portion of the way back to its starting (i.e. flat, horizontal) position and 207 performing the surgical procedure through said ‘working corridor’.

In at least one embodiment, the Supine/ATP Lumbar Support surgical method for approaching the spine of a patient for spinal interbody fusion device implant includes positioning a patient on an operating table in the supine position. It is appreciated that for certain patients, and in anticipation of the shift in forces as a result of a supine placement on the operating table, that one or more means for supporting the patient may be implemented. In at least one embodiment one or more supporting bolsters is used below the patient's lumbar or cervical spine to provide a means to facilitate desired sagittal plane curvature of the patient's spine.

One exemplary Supine/ATP Lumbar Support surgical method further includes mounting one or more retractor frames to the operating table on the patient's side, rotating the operating table about the patients' vertical (i.e. superior-inferior) axis such that one of the patient's sides is closer to the ceiling and the contralateral side is closer to the floor, performing an anterior-to-psoas surgical approach to the spine, placing one or more retractors in a position that creates a ‘working corridor’ to the spine, stabilizing the one or more retractors to a retractor frame, rotating the operating table about the patient's vertical axis at least a portion of the way back to its starting (i.e. flat, horizontal) position, and performing the surgical procedure through said ‘working corridor’. It should be appreciated that the mounting of the one or more retractor frames can occur just under the axilla and on the right side just above the knee of the patient in certain embodiments.

It should be understood that the above steps are not all inclusive, and additional steps may be implemented as the surgeon see fit. In at least one embodiment, normal draping and prep for surgery is completed after mounting the one or more retractor frame. In at least one embodiment, the incision made in the surgical procedure on the patient is approximately made at the anterior axillary line on the patient's left side at a level that aligns with the spinal level requiring treatment.

It should be further appreciated that many operating tables exist in the art. While the exemplary methods detailed herewithin may be performed on any operating table, for optimal results, the operating table should have the capabilities of being rotated to at least 25°.

Supine ATP with Buttock Bolster Method

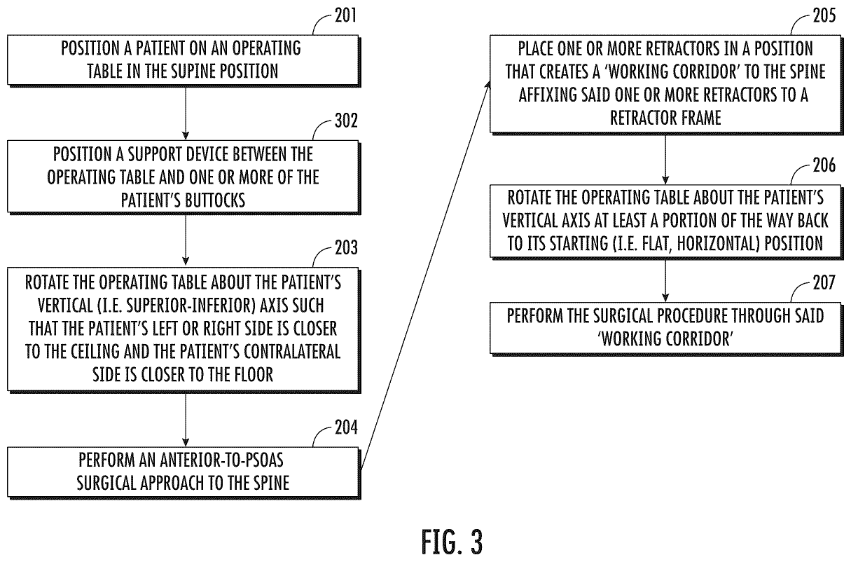

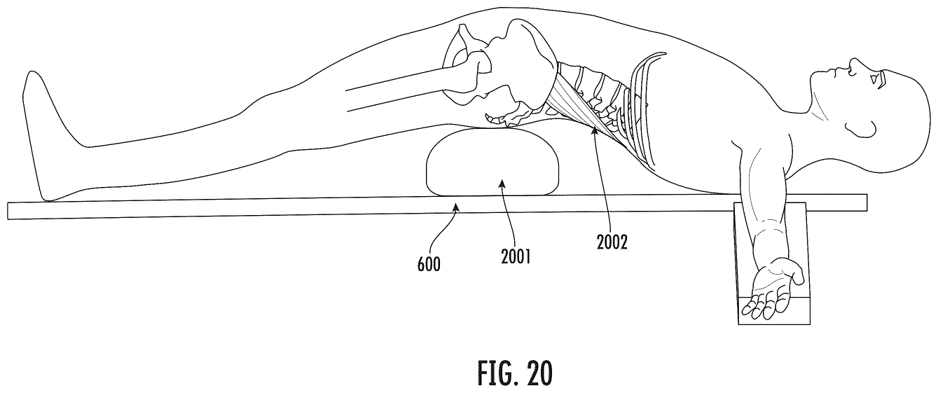

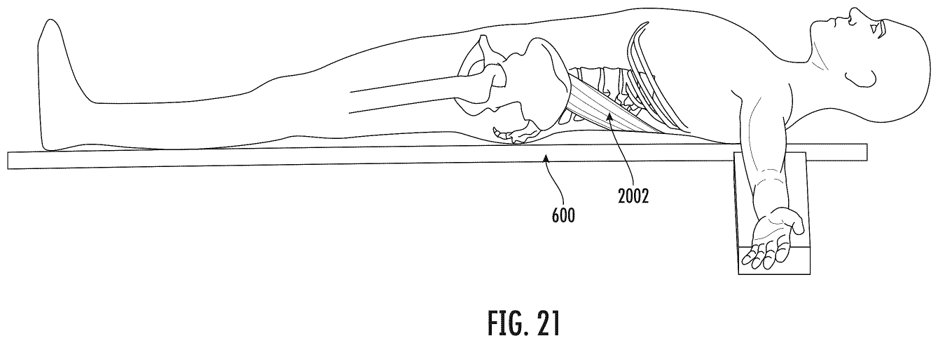

Embodiments of the present disclosure allow for inventive surgical methods to be used in association with one or more spinal interbody fusion or disc replacement device(s). It should be appreciated that such methods may be used with any spinal interbody fusion or disc replacement device(s), and nothing herein is intended to limit the surgical methods strictly to the use of the spinal interbody fusion devices disclosed herein. FIG. 3 illustrates at least one method of surgically approaching the spine of a patient for spinal intervertebral implantation with a buttock bolster. FIG. 20 illustrates a subject in position with a 2001 buttock bolster and the effect this position has on reducing the 2002 psoas muscle width. FIG. 21 shows a subject in position without a buttock bolster showing a loose 2002 psoas muscle.

As illustrated in FIG. 3, in at least one embodiment, the surgical method includes 201 positioning a patient on an operating table in the supine position, 302 positioning a support device between the operating table and the patient's buttocks, 203 rotating the operating table about the patients' vertical (i.e. superior-inferior) axis such that the patient's left or right side is closer to the ceiling and the patient's contralateral side is closer to the floor, 204 performing an anterior-to-psoas surgical approach to the spine, 205 placing one or more retractors in a position that creates a ‘working corridor’ to the spine affixing said one or more retractors to a retractor frame, 206 rotating the operating table about the patient's vertical axis at least a portion of the way back to its starting (i.e. flat, horizontal) position, and 207 performing the surgical procedure through said ‘working corridor’.

In at least one embodiment, the Supine/ATP Buttock Bolster surgical method for approaching the spine of a patient for spinal interbody fusion device implant includes positioning a patient on an operating table in the supine position. It is appreciated that for certain patients, and in anticipation of the shift in psoas muscle cross sectional area that can be affected by hyperextension of the hip joint, that one or more means for bolstering one or more of the patient's buttocks may be implemented. In at least one embodiment, one or more bolsters are used below the patient's buttock or buttocks to provide a means to facilitate desired transverse plane psoas muscle cross section.

One exemplary Supine/ATP Buttock Bolster surgical method further includes mounting one or more retractor frames to the operating table on the patient's side, rotating the operating table about the patients' vertical (i.e. superior-inferior) axis such that one of the patient's sides is closer to the ceiling and the contralateral side is closer to the floor, performing an anterior-to-psoas surgical approach to the spine, placing one or more retractors in a position that creates a ‘working corridor’ to the spine, stabilizing the one or more retractors to a retractor frame, rotating the operating table about the patient's vertical axis at least a portion of the way back to its starting (i.e. flat, horizontal) position, and performing the surgical procedure through said ‘working corridor’. It should be appreciated that the mounting of the one or more retractor frames can occur just under the axilla and on the right side just above the knee of the patient in certain embodiments.

It should be understood that the above steps are not all inclusive, and additional steps may be implemented as the surgeon see fit. In at least one embodiment, normal draping and prep for surgery is completed after mounting the one or more retractor frame. In at least one embodiment, the incision made in the surgical procedure on the patient is approximately made at the anterior axillary line on the patient's left side at a level that aligns with the spinal level requiring treatment.

It should be further appreciated that many operating tables exist in the art. While the exemplary method may be performed on any operating table, for optimal results, the operating table should have the capabilities of being rotated to at least 25°.

Supine ATP with Torso Tilt Method

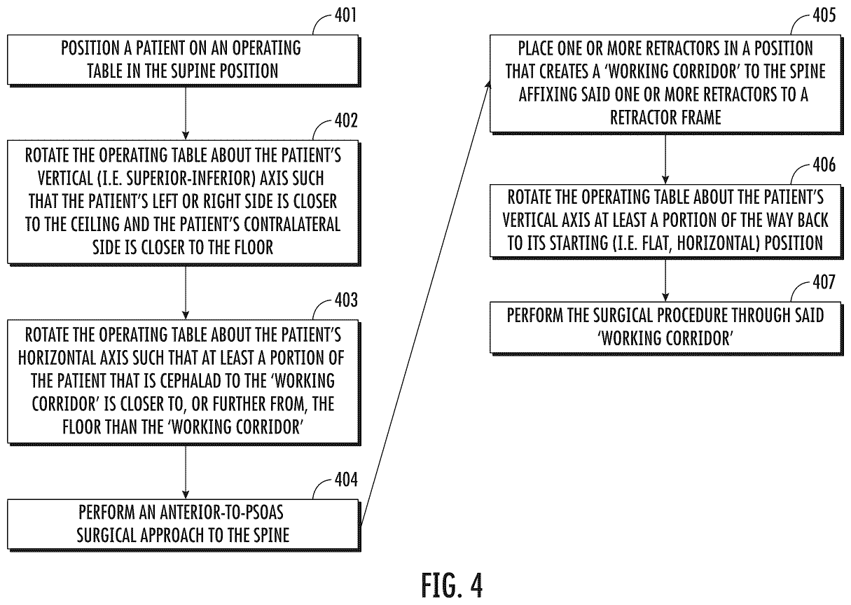

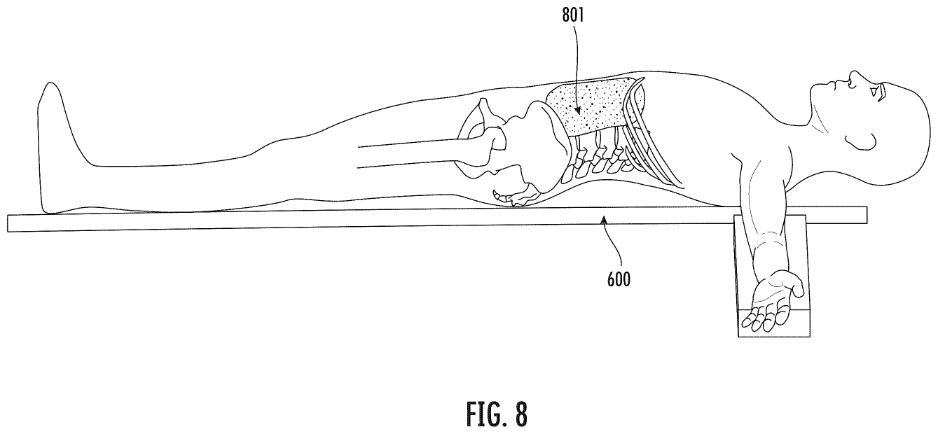

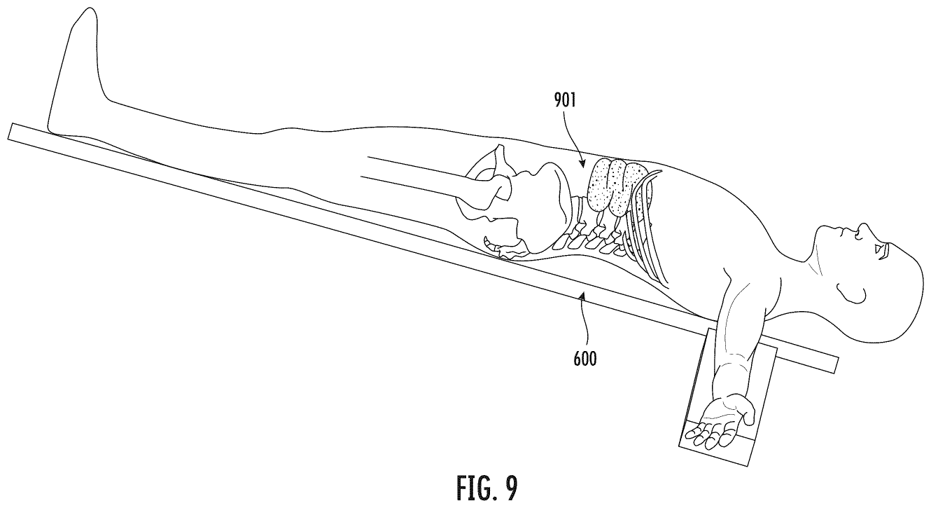

Embodiments of the present disclosure allow for inventive surgical methods to be used in association with one or more spinal interbody fusion or disc replacement device. It should be appreciated that such methods may be used with any spinal interbody fusion or disc replacement device, and nothing herein is intended to limit the surgical methods strictly to the use of the spinal interbody fusion devices disclosed herein. FIG. 4 illustrates at least one method of surgically approaching the spine of a patient for spinal intervertebral implantation with a torso tilt. As illustrated in FIG. 4, in at least one embodiment, the surgical method includes 401 positioning a patient on an operating table in the supine position, 402 rotating the operating table about the patients' vertical (i.e. superior-inferior) axis such that the patient's left or right side is closer to the ceiling and the patient's contralateral side is closer to the floor, 403 rotating the operating table about the patient's horizontal axis such that at least of portion of the patient that is cephalad to the ‘working corridor’ is closer to, or further from, the floor than the ‘working corridor’, 404 performing an anterior-to-psoas surgical approach to the spine, 405 placing one or more retractors in a position that creates a ‘working corridor’ to the spine affixing said one or more retractors to a retractor frame, 406 rotating the operating table about the patient's vertical axis at least a portion of the way back to its starting (i.e. flat, horizontal) position, and 407 performing the surgical procedure through said ‘working corridor’. FIG. 8 shows a 801 peritoneal sac of a patient in a level position on an 600 operating table prior to step 401 of FIG. 4. FIG. 9 shows a 901 mobilized sac of a patient in a tilted position on an 600 operating table after step 401 of FIG. 4.

In at least one embodiment, the Supine/ATP Torso Tilt surgical method for approaching the spine of a patient for spinal interbody fusion device implant includes positioning a patient on an operating table in the supine position. It is appreciated that for certain patients, and in anticipation of the shift in abdominal soft tissue position that can be affected by tilting the patient about the patient's x-axis, that a rotation of the patient about this axis may be implemented. In at least one embodiment, the patient's torso may be rotated about the x-axis to provide a means to facilitate desired abdominal soft tissue repositioning away from the operative corridor. While the term “Torso Tilt” is utilized in some instances throughout this disclosure, the position could be referred to as head down and, more commonly, the “Trendelenberg Position” with the feet elevated. Additionally, the position of the subject could be in the “Reverse Trendelenberg Position” with the head elevated.

One exemplary Supine/ATP Torso Tilt surgical method further includes mounting one or more retractor frames to the operating table on the patient's side, rotating the operating table about the patients' vertical (i.e. superior-inferior) axis such that one of the patient's sides is closer to the ceiling and the contralateral side is closer to the floor, performing an anterior-to-psoas surgical approach to the spine, placing one or more retractors in a position that creates a ‘working corridor’ to the spine, stabilizing the one or more retractors to a retractor frame, rotating the operating table about the patient's vertical axis at least a portion of the way back to its starting (i.e. flat, horizontal) position, and performing the surgical procedure through said ‘working corridor’. It should be appreciated that the mounting of the one or more retractor frames can occur just under the axilla and on the right side just above the knee of the patient in certain embodiments.

It should be understood that the above steps are not all inclusive, and additional steps may be implemented as the surgeon see fit. In at least one embodiment, normal draping and prep for surgery is completed after mounting the one or more retractor frame. In at least one embodiment, the incision made in the surgical procedure on the patient is approximately made at the anterior axillary line on the patient's left side at a level that aligns with the spinal level requiring treatment.

It should be further appreciated that many operating tables exist in the art. While the exemplary method may be performed on any operating table, for optimal results, the operating table should have the capabilities of being rotated to at least 25°.

Supine ATP with Angled Patient Positioner Method

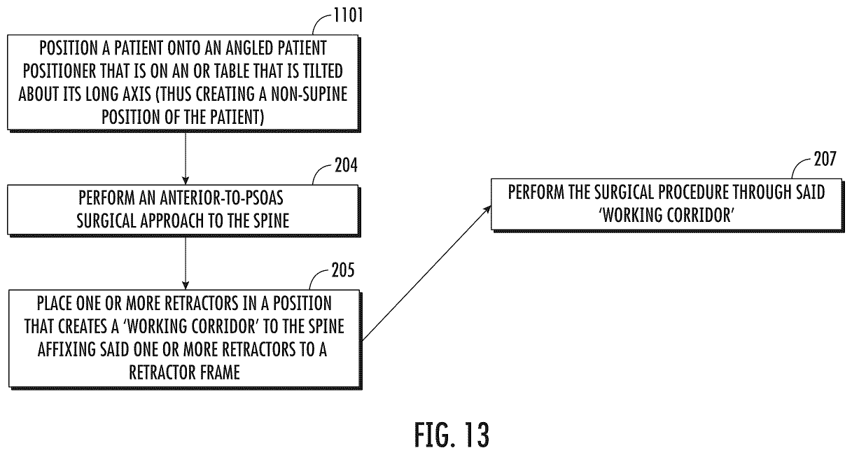

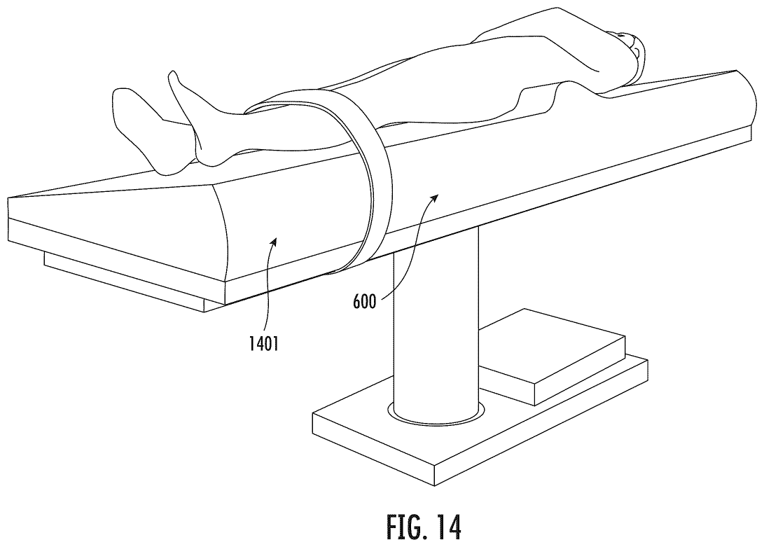

Embodiments of the present disclosure allow for inventive surgical methods to be used in association with one or more spinal interbody fusion or disc replacement device(s). It should be appreciated that such methods may be used with any spinal interbody fusion or disc replacement device(s), and nothing herein is intended to limit the surgical methods strictly to the use of the spinal interbody fusion devices disclosed herein. FIG. 13 shows a method of surgically approaching the spine of a patient for spinal intervertebral implantation without the need for any operating table rotation. As illustrated in FIG. 13, in at least one embodiment, the surgical method includes 1101 positioning a patient on to an angled patient positioner that is on an operating table that is wedge shape in cross section (thus creating a non-supine position of the patient), 204 performing an anterior-to-psoas surgical approach to the spine, 205 placing one or more retractors in a position that creates a ‘working corridor’ to the spine affixing said one or more retractors to a retractor frame, and 207 performing the surgical procedure through said ‘working corridor’. FIG. 14 illustrates at least one method of surgically approaching the spine of a patient for spinal intervertebral implantation with an 1401 Angled Patient Positioner using the surgical method of FIG. 13.

In at least one embodiment, the Supine/ATP Angled Patient Positioner surgical method for approaching the spine of a patient for spinal interbody fusion device implant includes positioning a patient on an operating table that is equipped with an Angled Patient Positioner. It is appreciated that the Angled Patient Positioner provides a means to facilitate the desired patient position during the surgical approach step of the method without the need for rotating the OR table.

One exemplary Angled Patient Positioner surgical method further includes mounting one or more retractor frames to the operating table on the patient's side, rotating the operating table about the patients' vertical (i.e. superior-inferior) axis such that the patient is oriented in a flat, horizontal position when performing the surgical procedure through said ‘working corridor’. It should be appreciated that the mounting of the one or more retractor frames can occur just under the axilla and on the right side just above the knee of the patient in certain embodiments.

It should be understood that the above steps are not all inclusive, and additional steps may be implemented as the surgeon see fit. In at least one embodiment, normal draping and prep for surgery is completed after mounting the one or more retractor frame. In at least one embodiment, the incision made in the surgical procedure on the patient is approximately made at the anterior axillary line on the patient's left side at a level that aligns with the spinal level requiring treatment.

It should be further appreciated that many operating tables exist in the art. While the exemplary method may be performed on any operating table, for optimal results, the operating table should have the capabilities of being rotated to at least 25°.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments.

It should be understood that various changes can be made in the function and arrangement of elements without departing from the scope as set forth in the appended claims and the legal equivalents thereof.

It should further be appreciated that while a certain arrangement of steps exist for one or more embodiments, steps from particular embodiments may be removed, or alternatively used in addition to the steps for the embodiments disclosed herein.

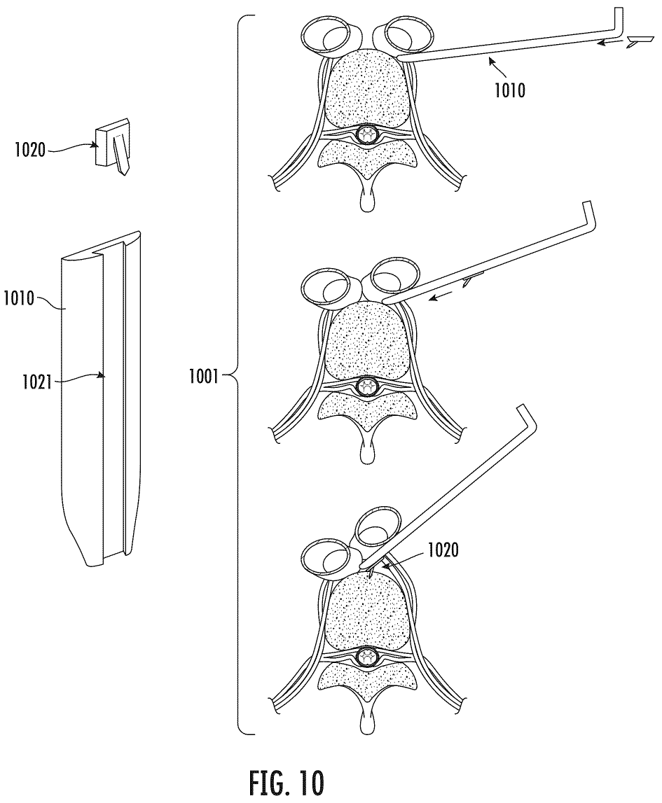

FIG. 10 illustrates at least one exemplary embodiment that maintains position without the need for a retractor frame. The embodiment of FIG. 10 illustrates at least one way to accomplish 1001 surgery without the need for a retractor frame. As shown in FIG. 10 at least one ‘stand alone’ 1010 retractor maintains its position because it has a 1020 ‘spline’ that anchors it in place in the disc. The 1021 groove in the back of the 1010 retractor is configured such that it allows the 1020 spline to slide along the 1010 retractor. This embodiment obviates the need for a retractor frame. Additionally, even more simply, a hand-held retractor without a spline could be used. A hand-held retractor has an advantage of enabling the surgeon to periodically relieve the pressure acting on the vessel during the procedure to reduce the risk of life-threatening vascular complications (e.g. arterial thrombosis). One exemplary Supine/ATP surgical method involving this embodiment can include rotating the operating table about the patients' vertical (i.e. superior-inferior) axis such that one of the patient's sides is closer to the ceiling and the contralateral side is closer to the floor, performing an anterior-to-psoas surgical approach to the spine, placing one or more retractors in a position that creates a ‘working corridor’ to the spine, stabilizing the one or more retractors to the patient's disc or vertebra or stabilizing it using another means, such as manually holding the retractor, rotating the operating table about the patient's vertical axis at least a portion of the way back to its starting (i.e. flat, horizontal) position, and performing the surgical procedure through said ‘working corridor’. It should be appreciated that the stabilizing of the one or more retractors can occur above and/or below the spinal level that is being operated on in the patient in certain embodiments.

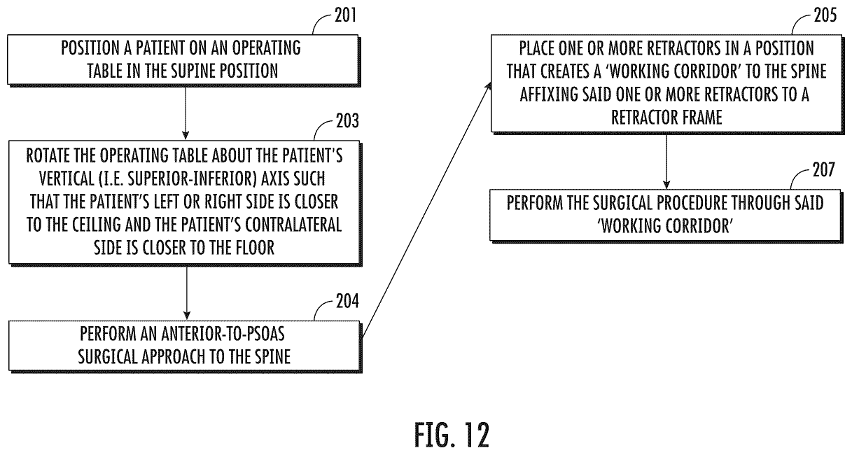

In another embodiment, a mat can be placed under the supine patient during patient positioning. This mat generally includes two substantially parallel inflatable bolsters; one under the buttocks and one under the low back. During the approach, the bolster under the buttocks is inflated to help provide access to the disc. One reason the bolster helps access the disc is because this position creates a stretch in the psoas muscle which reduces its cross section (see FIG. 17). This reduced cross section gives the surgeon more space to access the spine. Then, during the placement of the cage, the buttock bolster is deflated and the lumbar bolster is inflated. The lumbar bolster improves lordosis and also helps keep the subject/patient from sliding on the table. Additionally, the patient's head is tilted down. FIG. 12 illustrates at least one method of surgically approaching the spine of a patient for spinal intervertebral implantation with the patient in the tilted position during the final steps of the procedure. As illustrated in FIG. 12, in at least one embodiment, the surgical method includes 201 positioning a patient on an operating table in the supine position, 203 rotating the operating table about the patients' vertical (i.e. superior-inferior) axis such that the patient's left or right side is closer to the ceiling and the patient's contralateral side is closer to the floor, 204 performing an anterior-to-psoas surgical approach to the spine, 205 placing one or more retractors in a position that creates a ‘working corridor’ to the spine affixing said one or more retractors to a retractor frame, and 207 performing the surgical procedure through said ‘working corridor’.

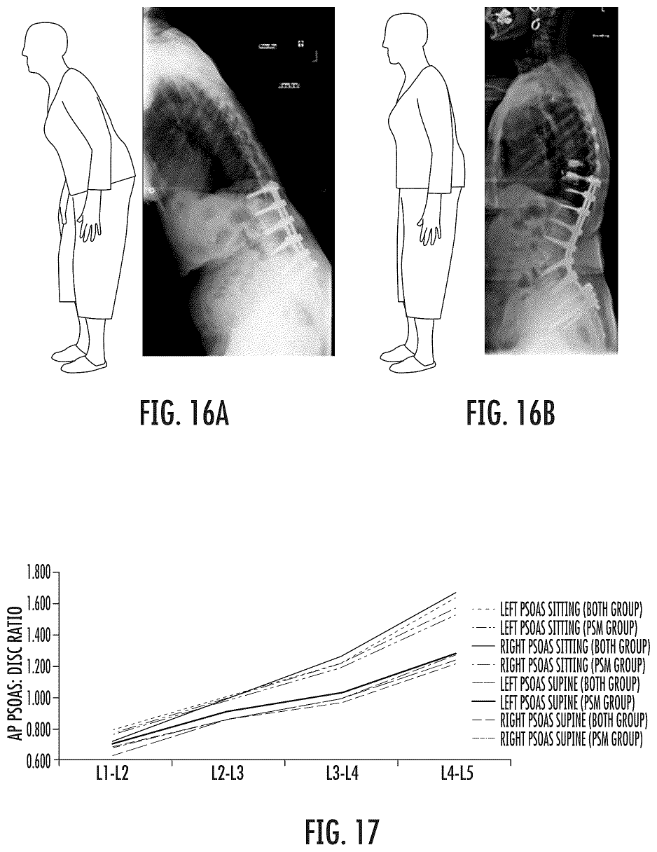

In at least one aspect, the exemplary apparatus, system(s), and method(s) detailed herein provide simpler, more effective methods of using the ‘Anterior to Psoas’ (ATP) surgical approach corridor and reduce surgical risks. In one exemplary aspect, a Supine/ATP with Lumbar Support Method is provided that includes advantages over prior art (i.e. with no lumbar support with the patient in the supine position): lumbar support provides the spine surgeon with an easier way to maintain and/or reconstruct the proper sagittal alignment (i.e. lordotic curvature). Without a lumbar support device gravity tends to flatten the lumbar spine while the patient is supine on the OR table. FIG. 16a shows a subject in position with a flattened spine. If a patient is fused with a flattened spine, they have difficulty standing upright (see FIG. 16a). One exemplary study found a significant effect on lordosis as a function of patient positioning (Smith, Tyler G., et al. “Effects of Surgical Positioning on L4-L5 Accessibility and Lumbar Lordosis in Lateral Transpsoas Lumbar Interbody Fusion: A Comparison of Prone and Lateral Decubitus in Asymptomatic Adults”, World Neurosurgery 149 (2021): e705-e713.) Comparatively, and as illustrated in FIG. 16b, a patient fused with a flattened spine (as illustrated in FIG. 16a) can restored to normal posture due a realignment operation to restore proper alignment.

A lumbar bolster also serves to reduce patient movement relative to the OR table while it is repositioned. In one exemplary aspect, a Supine/ATP with Buttock Bolster Method is provided that includes advantages over prior art (no buttock bolster with the patient in the supine hip extended position): a buttock bolster hyper extends the hip joint which, in turn, extends the Psoas muscle which reduces its cross section as compared to its cross section with the patient in a hip flexed position. A smaller cross section reduces the risk of iatrogenic injury to this muscle during the development of the spinal approach corridor. This exemplary study found the effect illustrated in FIG. 17 (Buckland, Aaron J., et al. “Psoas morphology differs between supine and sitting magnetic resonance imaging lumbar spine: implications for lateral lumbar interbody fusion”, Asian spine journal 12.1 (2018): 29.)

In one exemplary aspect, a Supine/ATP with Trendelenberg Position and Reverse Trendelenberg Position is provided that includes advantages over prior art (i.e. no Trendelenberg Position or Reverse Trendelenberg Position with the patient in only the supine fully horizontal position): abdominal contents require less displacement during the establishment of the approach corridor when the patient is put in a Trendelenberg Position or Reverse Trendelenberg Position. This reduces the risk of iatrogenic injury of the peritoneum. It also reduces the amount of retraction pressure required to dock the retractors relative to the operative site on the spine.

In one exemplary aspect, a Supine/ATP with Lumbar Support Method with Supine/ATP with Buttock Bolster Method with Supine/ATP with Trendelenberg Position and Reverse Trendelenberg Position is provided that includes advantages over prior art: the combination of all of the above advantages.

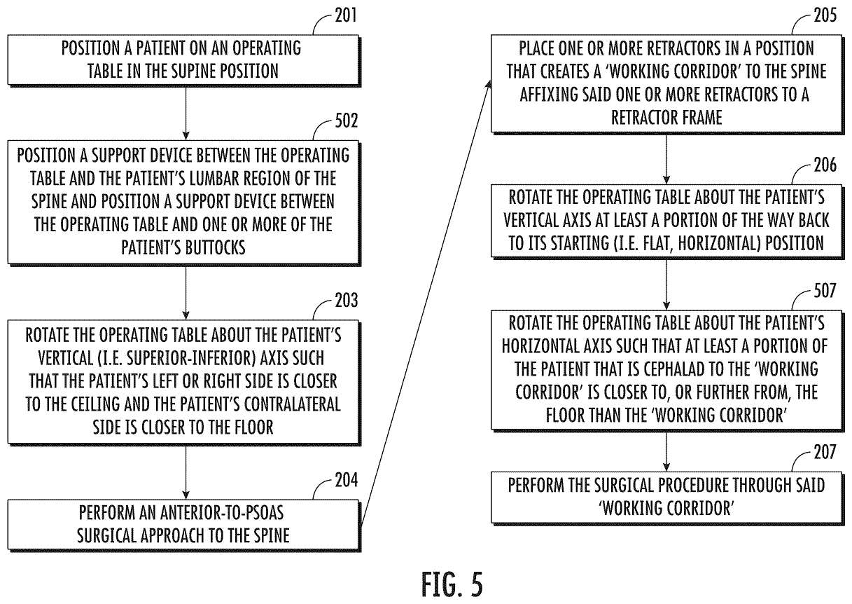

In at least one embodiment, each of the lumbar support, buttock bolster, and torso tilt may be used individually or in combination in certain surgical methods. FIG. 5 illustrates at least one method of surgically approaching the spine of a patient for spinal intervertebral implantation with a lumbar support, buttock bolster, and torso tilt. As illustrated in FIG. 5, in at least one embodiment, the surgical method includes 201 positioning a patient on an operating table in the supine position, 502 positioning a support device between the operating table and the patient's lumbar region of the spine and position a support device between the operating table and one or more of the patient's buttocks, 203 rotating the operating table about the patients' vertical (i.e. superior-inferior) axis such that the patient's left or right side is closer to the ceiling and the patient's contralateral side is closer to the floor, 204 performing an anterior-to-psoas surgical approach to the spine, 205 placing one or more retractors in a position that creates a ‘working corridor’ to the spine affixing said one or more retractors to a retractor frame, 206 rotating the operating table about the patient's vertical axis at least a portion of the way back to its starting (i.e. flat, horizontal) position, 507 rotating the operating table about the patient's horizontal axis such that at least of portion of the patient that is cephalad to the ‘working corridor’ is closer to, or further from, the floor than the ‘working corridor’, and 207 performing the surgical procedure through said ‘working corridor’.

In one exemplary aspect, a Supine/ATP with Parallel Inflatable/Deflatable Bolsters Method is provided that includes advantages over prior art (no inflatable/deflatable bolsters): the ability to reduce the psoas muscle cross section during one phase of the surgery (retractor docking during the establishment of the approach corridor) and then later re-establish the normal psoas muscle cross section to ensure the patient's lordotic angle is optimal for fusion can only be done in a cumbersome manner of installing and uninstalling independent bolsters. This cannot be accomplished without violating the sterile operative field. Doing so, extends surgical times and patient risks.

In one exemplary aspect, a Supine/ATP with Standalone Retractor Method is provided that includes advantages over prior art (i.e. fixed frame retractor method): standalone retractors that affix to the patient's spine are less cumbersome to setup and use and less expensive than retractors that require a frame that attaches to the OR table.

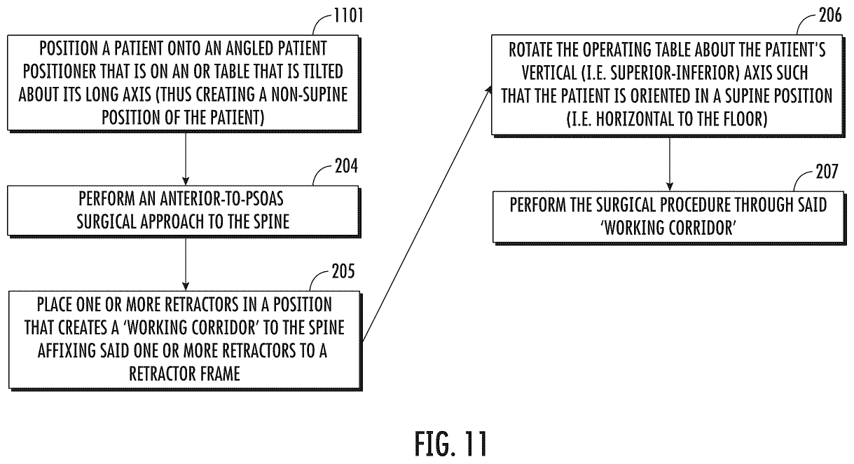

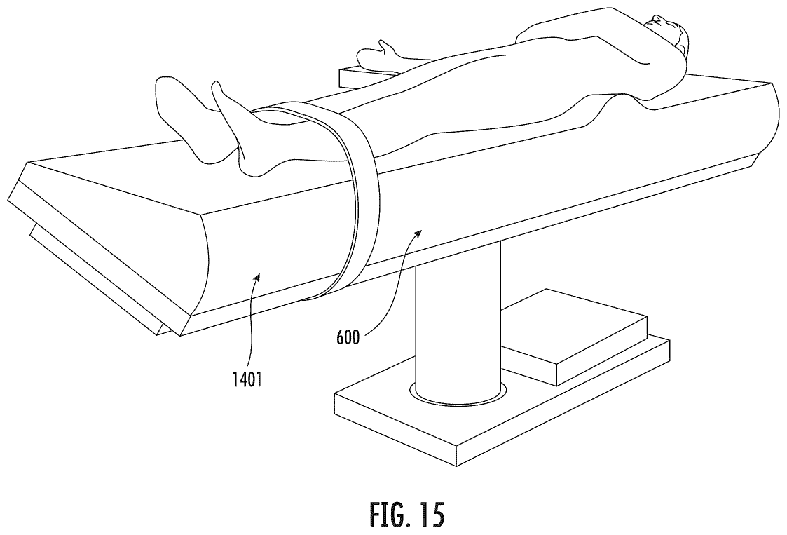

In one exemplary aspect, a Supine/ATP with Angled Patient Positioner Method is provided that includes advantages over prior art (i.e. no Angled Patient Positioners): the ability to position the patient during one phase of the surgery without having to rotate the OR table to cause one side of the patient to be closer to the ceiling than the other side of the patient. This reduces a step in the procedure thereby reducing OR time. Furthermore, means for preventing the patient from sliding on the Angled Patient Positioner such as one or more circumferential straps can be incorporated to eliminate the cumbersome manner of installing and uninstalling independent bolsters. This cannot be accomplished without violating the sterile operative field. Doing so, extends surgical times and patient risks. FIG. 11 illustrates at least one method of surgically approaching the spine of a patient for spinal intervertebral implantation with an angled patient positioner. FIGS. 14-15 show a subject at two positions with an angled patient positioner. As illustrated in FIG. 11, in at least one embodiment, the surgical method includes 1101 positioning a patient on to an angled patient positioner that is on an operating table that is tilted about its long axis (thus creating a non-supine position of the patient), 204 performing an anterior-to-psoas surgical approach to the spine, 205 placing one or more retractors in a position that creates a ‘working corridor’ to the spine affixing said one or more retractors to a retractor frame, 206 rotating the operating table about the patient's vertical axis at least a portion of the way back to its starting (i.e. flat, horizontal) position, and 207 performing the surgical procedure through said ‘working corridor’. FIG. 14 illustrates at least one method of surgically approaching the spine of a patient for spinal intervertebral implantation with an 1401 Angled Patient Positioner using the surgical method of FIG. 11.

The foregoing description is illustrative of particular embodiments of the disclosure, but is not meant to be a limitation upon the practice thereof. The following claims, including all equivalents thereof, are intended to define the scope of the disclosure.

1. A method of performing spinal surgical procedures, the method comprising:
(a) positioning a patient on an operating table in the supine position;
(b) rotating the operating table about the patients' vertical (i.e. superior-inferior) axis such that the patient's side is closer to the ceiling and the contralateral patient's side is closer to the floor;
(c) performing an anterior-to-psoas surgical approach to the spine;
(d) placing one or more retractors in a position that creates a ‘working corridor’ to the spine;
(e) stabilizing said one or more retractors to a retractor frame; and
(f) performing the surgical procedure through said ‘working corridor’.

2. The method of claim 1 further comprising positioning a support device between the operating table and the patient's lumbar region of the spine between steps (a) and (b).

3. The method of claim 1 further comprising positioning a support device between the operating table and one or more of the patient's buttocks between steps (a) and (b).

4. The method of claim 2 further comprising rotating the operating table about the patient's vertical axis at least a portion of the way back to its starting (i.e. flat, horizontal) position after the completion of step (e).

5. The method of claim 1 further comprising rotating the operating table about the patient's horizontal axis such that at least of portion of the patient that is cephalad to the ‘working corridor’ is closer to, or further from, the floor than the ‘working corridor’ prior to the completion of step (c).

6. The method of claim 1 wherein the rotation of the operating table of step (b) is rotated at least 25°.

7. The method of claim 1 wherein the surgical procedure of step (f) includes implantation of an intervertebral implant is an interbody fusion device or a disc replacement device.

8. A method of performing spinal surgical procedures, the method comprising:
(a) positioning a patient on an operating table such that the patient's side is closer to the ceiling and the contralateral patient's side is closer to the floor;
(b) performing an anterior-to-psoas surgical approach to the spine;
(c) placing one or more retractors in a position that creates a ‘working corridor’ to the spine;
(d) stabilizing said one or more retractors to a retractor frame; and
(e) performing the surgical procedure through said ‘working corridor’.

9. The method of claim 8 further comprising positioning a support device between the operating table and the patient's lumbar region of the spine prior to the completion of step (b).

10. The method of claim 8 further comprising positioning a support device between the operating table and one or more of the patient's buttocks prior to the completion of step (b).

11. The method of claim 8 further comprising rotating the operating table about the patient's vertical axis after the completion of step (d) and prior to the completion of step (e).

12. The method of claim 8 further comprising rotating the operating table about the patient's vertical axis at least a portion of the way towards a supine position of the patient after the completion of step (d).

13. The method of claim 8 further comprising rotating the operating table about the patient's horizontal axis such that at least of portion of the patient that is cephalad to the ‘working corridor’ is closer to, or further from, the floor than the ‘working corridor’ after the completion of step (a).

14. The method of claim 11 wherein the rotation of the operating table is rotated at least 25°.

15. The method of claim 8 wherein the surgical procedure of step (e) includes implantation of an intervertebral implant is an interbody fusion device or a disc replacement device.

16. The method of claim 8 in which the patient is positioned on an angled patient positioner on an operating table in step (a) thus creating a non-supine position of the patient.

17. A method of performing spinal surgical procedures, the method comprising:
(a) positioning a patient on an operating table in the supine position;
(b) rotating the operating table about the patients' vertical (i.e. superior-inferior) axis such that the patient's side is closer to the ceiling and the contralateral patient's side is closer to the floor;
(c) rotating the operating table about the patient's horizontal axis such that at least of portion of the patient that is cephalad to the ‘working corridor’ is closer to, or further from, the floor than the ‘working corridor’; and
(d) performing an anterior-to-psoas surgical approach to the spine;
(e) placing one or more retractors in a position that creates a ‘working corridor’ to the spine;
(f) stabilizing said one or more retractors to a retractor frame;
(g) rotating the operating table about the patient's vertical axis at least a portion of the way back to its starting (i.e. flat, horizontal) position; and
(h) performing the surgical procedure through said ‘working corridor’.

18. The method of claim 17 further comprising positioning a support device between the operating table and the patient's lumbar region of the spine between steps (a) and (b).

19. The method of claim 17 further comprising positioning a support device between the operating table and one or more of the patient's buttocks between steps (a) and (b).

20. A method of performing spinal surgical procedures, the method comprising:
a. positioning a patient on an operating table in the supine position;
b. rotating the operating table about the patients' vertical (i.e. superior-inferior) axis such that the patient's side is closer to the ceiling and the contralateral patient's side is closer to the floor,
c. performing an anterior-to-psoas surgical approach to the spine;
d. placing one or more retractors in a position that creates a ‘working corridor’ to the spine;
e. stabilizing said one or more retractors; and
f. performing the surgical procedure through said ‘working corridor’.
